(19) United States
(12) Patent Application Publication
(10) Pub. No.: US 2026/0060855 A1
(43) Pub. Date: Mar. 5, 2026

(54) PORTABLE EMERGENCY RESPONSE KITS WITH INTEGRATED SIGNAL TRANSMISSION SYSTEM

(71) Applicant: FFF Enterprises, Inc., Temecula, CA (US)

(72) Inventors: Jesper Jensen, Temecula, CA (US); Brandon Beckendorf, Temecula, CA (US)

(21) Appl. No.: 19/312,483

(22) Filed: Aug. 28, 2025

Related U.S. Application Data
(60) Provisional application No. 63/689,265, filed on Aug. 30, 2024.

(51) Int. Cl. A61F 17/00; G16H 40/63

(52) U.S. Cl. CPC A61F 17/00; G16H 40/63

(57) ABSTRACT

A portable emergency response kit designed for rapid intervention in opioid overdose or cardiac arrest scenarios comprises a durable case with compartments for specific medical devices, such as opioid overdose treatment medication or an Automated External Defibrillator (AED). The case features a breakable closed circuit that is, upon opening, triggered to send a signal to notify emergency medical services (EMS) or predefined contacts of the kit's location.

The system utilizes GPS module and may include dual-mode communication capabilities for reliable alert transmission in various environments. Additional features may include a transparent case material for visual confirmation of contents, tamper-evident seals, QR codes linked to instructional content, and a battery power source. The kit is designed to be user-friendly, incorporating ergonomic elements and optional accessories for enhanced portability and storage, ensuring rapid and effective response in critical situations.

### CROSS REFERENCE TO RELATED APPLICATIONS

This application claims the benefit of priority to U.S. Provisional Application No. 63/689,265, filed on Aug. 30, 2024. The entire contents of this patent application is herein incorporated by reference.

### TECHNICAL FIELD

This disclosure relates to the field of emergency medical devices and systems, specifically portable emergency response kits equipped with signal transmission technology to automatically notify emergency medical services (EMS) or predefined contacts of the kit's location upon activation. The disclosure addresses, for example, opioid overdose response and automated external defibrillator (AED) usage, incorporating integrated communication and alert systems for timely medical intervention.

### BACKGROUND OF THE DISCLOSURE

Opioid overdose and cardiac arrest are leading causes of sudden death worldwide, necessitating immediate medical intervention to improve survival rates. For example, naloxone is widely recognized as an effective treatment for reversing opioid overdoses, while Automated External Defibrillators (AEDs) are crucial for addressing cardiac arrest. Both treatments are time-sensitive and rely on rapid access to emergency medical services or nearby responders.

Current emergency response kits, such as those containing naloxone or AEDs, are often limited in their ability to alert emergency responders immediately upon deployment. Typically, users must manually call for help or notify others of the situation, which can cause critical delays, particularly when the user is alone or incapacitated.

Moreover, conventional kits or systems may lack integrated location-based technologies, further complicating the response time. In addition, existing kits may not include features to prevent tampering or ensure proper use by untrained individuals.

Therefore, there is a need for a portable emergency response kit that can automatically and reliably notify emergency medical services and/or predefined contacts upon being accessed. Such a kit should provide location identifying data, be easy to deploy, and minimize delays in emergency response, thereby enhancing the chances of survival and recovery in, for example, overdose or cardiac arrest scenarios.

### SUMMARY OF THE DISCLOSURE

In some embodiments, a portable opioid overdose response kit comprises a case having a compartment configured to contain an opioid overdose treatment medication (e.g., naloxone), a breakable closed circuit configured to be triggered upon opening the case, wherein opening the case breaks the circuit, and the broken circuit is triggered to send a signal to a communication device external to the kit.

The compartment may contain an opioid overdose treatment medication (e.g., naloxone). Opening the case breaks the circuit, which triggers the kit to send a signal to notify emergency medical services (EMS) or a predefined contact list. The case may be made of transparent material that allows visual confirmation of the contents of the case. The kit may further comprise a QR code linked to instructions for administering an opioid overdose treatment medication (e.g., naloxone). The kit may comprise a GPS module to transmit the location of the kit. The kit may further comprise a battery power source. The case may comprise an upper compartment and a lower compartment, and the breakable closed circuit may be configured to be triggered upon separating the upper compartment from the lower compartment, wherein separating the compartments breaks the circuit, and the broken circuit is triggered to send the signal to the communication device external to the kit. The circuit may be housed by a board or pad with an adhesive backing, which is integrated into the case (e.g., attached to the case). In some embodiment, the kit is configured transmit a signal when pad or board (or other physical) structure is torn when the case is opened.

In some embodiments, a portable Automated External Defibrillator (AED) kit comprises a case having a compartment configured to contain an AED, a breakable closed circuit configured to be triggered upon opening the case, wherein opening the case breaks the circuit, and the broken circuit is triggered to send a signal to a communication device external to the kit.

The compartment may contain an Automated External Defibrillator (AED). Opening the case breaks the circuits, sending a signal to send an alert to a predefined contact list. The case may be made of transparent material that allows visual confirmation of the contents of the case. The kit may further comprise a QR code linked to instructions for operating the AED. The kit may comprise a GPS module to transmit the location of the kit. The kit may further comprise a battery power source. The case may comprise an upper compartment and a lower compartment, and the breakable closed circuit may be configured to be triggered upon separating the upper compartment from the lower compartment, wherein separating the compartments breaks the circuit, and the broken circuit is triggered to send a signal to a communication device external to the kit. The breakable closed circuit may be housed by a board or pad with an adhesive backing, which is integrated into the case.

In some embodiments, a portable emergency response kit comprises a case having an upper compartment and a lower compartment, a breakable closed circuit configured to be triggered upon separating the upper compartment from the lower compartment, wherein separating the compartments breaks the circuit, which triggers the kit to send a signal to a communication device external to the kit. The breakable closed circuit may be housed by a board or pad with an adhesive backing, which is integrated into the case.

### BRIEF DESCRIPTION OF DRAWINGS

The following figures and descriptions provide a detailed understanding of various embodiments of the present disclosure. The drawings illustrate the components and operation of portable emergency response kits. These example embodiments are intended to aid in understanding the inventive concepts and are not to be construed as limiting the scope of the disclosure.

FIG. 1 is a schematic diagram illustrating the portable opioid overdose emergency response kit in its closed state according to an example embodiment of the present invention.

FIG. 2 is a schematic diagram illustrating the portable opioid overdose emergency response kit in its open state according to an example embodiment of the present invention.

### DETAILED DESCRIPTION

The following detailed description of various embodiments of the disclosure is provided to illustrate specific configurations and implementations. It is not intended to be exhaustive or to limit the disclosure to the precise forms disclosed. Numerous modifications and variations are possible in light of the following teachings. The embodiments were chosen and described in order to best explain the principles of the disclosure and its practical applications, thereby enabling others skilled in the art to understand the disclosure for various embodiments and with various modifications that are suited to the particular use contemplated.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. These terms are merely intended to distinguish one component from another component, and the terms do not limit the nature, sequence, or order of the constituent components. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Throughout the specification, unless explicitly described to the contrary, the word “comprise” and variations such as “comprises” or “comprising” will be understood to imply the inclusion of stated elements but not the exclusion of any other elements. In addition, the terms “unit,” “-er,” “-or,” and “module” described in the specification mean units for processing at least one function and operation and can be implemented by hardware components or software components and combinations thereof.

Example embodiments are described as using a plurality of units to perform the example process, it is understood that the example processes may also be performed by one or a plurality of modules. Additionally, it is understood that the term controller/control unit refers to a hardware device that includes a memory and a processor and is specifically programmed to execute the processes described herein. The memory is configured to store the modules and the processor is specifically configured to execute said modules to perform one or more processes which are described further below.

Further, the control logic of the present disclosure may be embodied as non-transitory computer-readable media on a computer-readable medium containing executable program instructions executed by a processor, controller, or the like. Examples of computer-readable media include, but are not limited to, ROM, RAM, compact disc (CD)-ROMs, magnetic tapes, floppy disks, flash drives, smart cards, and optical data storage devices. The computer-readable medium can also be distributed in network-coupled computer systems so that the computer-readable media is stored and executed in a distributed fashion, e.g., by a telematics server or a Controller Area Network (CAN).

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

In the context of the present disclosure, the use of terms such as “may,” “might,” “can,” “could,” “would,” “should,” “suggest,” and similar language should be understood as being generally permissive, indicating that the actions or conditions described are within the scope of the disclosure, though not necessarily required.

The descriptions provided herein should not be construed as limiting but as example. Various changes and modifications may be made without departing from the spirit and scope of the disclosure. The claims should be construed broadly to cover all such variations, modifications, and equivalents within the scope of the disclosure.

As used herein, the term “transparent” refers to a material or surface that allows the passage of light such that objects, contents, or items behind or within it are visible to an observer. The degree of transparency may vary, but the material should be sufficiently clear to permit visual confirmation of the contents of the case without opening or altering it. Transparent materials may include, but are not limited to, clear plastics (such as polycarbonate, acrylic, or polyethylene terephthalate), glass, or other substances with similar light-transmitting properties.

As used herein, the term “closed circuit” refers to an electrical circuit configuration that remains in a closed (or complete) state under normal conditions, enabling the flow of electric current. The closed circuit is designed to be disrupted or opened when a specific event occurs, such as the opening or separation of a case or compartment, thereby interrupting the current flow. This disruption triggers an associated action, such as sending a signal to notify emergency medical services (EMS) and/or predefined contacts of the kit's location. The closed circuit may include, but is not limited to, conductive traces, breakable connections, switches, or other components configured to detect a change in the circuit's continuity and initiate a predetermined response.

As used herein, the term “opioid overdose treatment medication” refers to any pharmaceutical agent specifically formulated to counteract the life-threatening effects of an opioid overdose. This medication rapidly binds to opioid receptors in the central nervous system, displacing opioids and reversing symptoms such as respiratory depression, sedation, and loss of consciousness caused by an overdose. The most commonly used opioid overdose treatment medication is naloxone, which is available in various forms, including intranasal sprays, intramuscular injections, and intravenous formulations. This term may also encompass any future-developed medications with similar properties and effects designed to treat or reverse opioid overdoses.

The present disclosure relates to portable emergency response kits designed for rapid deployment in medical emergencies, such as opioid overdoses and cardiac arrest situations. These kits are configured to automatically notify emergency medical services (EMS) and/or predefined contacts of the kit's location upon activation, thereby improving the speed and effectiveness of emergency response.

The portable emergency response kit is designed to deliver rapid and effective support in life-threatening situations, including, but not limited to, opioid overdoses and cardiac emergencies. The kit may comprise a case with one or more compartments specifically designed to securely contain critical life-saving devices and medications, such as, but not limited to, opioid overdose treatment medications (like naloxone, naltrexone, or buprenorphine-naloxone combinations) or an Automated External Defibrillator (AED). The inclusion of these diverse treatment tools allows the kit to provide comprehensive emergency assistance for a wide range of medical crises.

### Case Design and Materials

The case may be constructed from durable, lightweight materials that offer superior protection against impacts, moisture, and extreme temperatures. These materials, which may include high-density polyethylene (HDPE), polypropylene (PP), or polycarbonate (PC), are chosen for their robustness, chemical resistance, and ability to withstand harsh environmental conditions. The case may also feature a transparent section or be entirely made from clear materials such as acrylic or polycarbonate, allowing for immediate visual confirmation of its contents. This transparency ensures that the life-saving devices or medications are visibly present, intact, and ready for immediate use, providing reassurance to medical professionals and individuals carrying the kit that the contents are accessible and in optimal condition.

### Closed Circuit Activation Mechanism

The kit may include a breakable closed circuit designed to trigger an alert to emergency responders when the case is opened, enhancing the likelihood of a timely response. This closed circuit may be implemented in various ways to ensure reliable activation while maintaining the compact and portable nature of the kit.

For example, a transistor and power source can be arranged within the circuit such that the transistor switches on and allows current to flow to a transmitter. This occurs when a conductor wire in the circuit is opened, causing a change in voltage across the transistor's terminals. This change in voltage activates the transmitter, which then sends a signal to notify emergency medical services (EMS) or predefined contacts.

Some embodiments may utilize a series of conductive traces, or a thin conductive film integrated along the opening seam between the upper and lower compartments of the case. These traces or films in the form of a seal are connected along one edge to form a hinged or foldable structure, allowing for easy access to the contents while maintaining structural integrity. These conductive elements may be constructed as printed circuits or flexible strips embedded within the material of the case. When the case is closed, these traces complete an electrical circuit; however, upon opening the case, the alignment of these traces in the form of a seal is disrupted, or the traces themselves may be physically broken, instantly interrupting the circuit. This disruption may trigger the closed circuit to send an alert signal to emergency responders, ensuring fully automatic activation without any additional action required from the user. To enhance reliability, this design may incorporate a redundant sensor or a secondary circuit to prevent false alarms due to minor impacts or vibrations.

In some embodiments, the kit may employ a pull-tab or tear-strip mechanism that incorporates conductive material and serves as a part of the circuit. This strip, positioned strategically to be the first point of interaction when accessing the kit, may function similarly to the opening mechanism of a cereal box. When the user pulls or tears the strip to open the kit, the conductive pathway integrated into the strip may be severed, breaking the circuit, and sending a signal. To further enhance the reliability of this activation method, the pull-tab may be designed with multiple conductive layers or tracks, ensuring the circuit breaks consistently regardless of the tear angle or the force applied. In addition, the pull-tab may be connected to a spring-loaded switch or micro-switch to add an extra layer of certainty that the signal is triggered immediately upon access, regardless of the user's handling speed or method.

In some embodiments, the kit may utilize a capacitor-driven signal activation mechanism, which leverages an energy storage component, such as a capacitor, to ensure reliable signal transmission even in low-power or no-power situations. While the case remains closed, the capacitor may charge from a small battery or external power source, maintaining a stored electrical charge. The capacity may be pre-charged. The circuit may include additional components, such as resistors, diodes, or a signal relay, to control and regulate the flow of current. When the case is opened, the primary circuit may break, and the stored charge in the capacitor may be instantly released through an auxiliary circuit designed to activate a transmitter or signal module. This capacitor-driven mechanism may allow for a controlled release of energy, ensuring that even if the primary power source fails, the signal is reliably transmitted to emergency responders. This design may also incorporate a feedback loop to confirm that the signal has been successfully sent, providing an added layer of assurance in critical situations.

Another possible configuration may involve a port-connected circuit, where the case features a series of conductive ports or contact points along the edges where the upper and lower compartments meet. When the case is closed, these ports may align precisely, completing an electrical circuit through magnetic contacts, spring-loaded pins, or conductive pads. As soon as the case is opened, the physical connection between these ports may be disrupted, breaking the circuit, and activating signal transmission. To ensure reliability, this mechanism may be designed with multiple redundant connections or interlocking ports to minimize the risk of false activations due to minor misalignments or impacts. The use of magnetic connectors or gold-plated contacts may further improve the durability and reliability of the connection points, ensuring consistent performance over time and in various environmental conditions.

The kit may be designed to immediately notify Emergency Medical Services (EMS) and other predefined contacts of the kit's location and use upon circuit disruption. The closed circuit may include a microcontroller or dedicated communication module equipped with one or more communication capabilities, such as cellular (GSM/4G/5G), Bluetooth, Wi-Fi, low-power wide-area networks (LPWAN), or other digital or analog signal.

The closed circuit may be connected to another circuit that includes the microcontroller or the communication module. In some configurations, a transistor and power source can be arranged to ensure that when a conductor wire in the circuit is broken, the change in voltage across the transistor's terminals causes the transmitter to activate and send the signal. The system is designed to detect circuit disruption automatically and may trigger a multi-channel communication protocol, which prioritizes available networks to ensure the fastest and most reliable connection. The system may dynamically switch between cellular, Bluetooth, or Wi-Fi based on signal strength, availability, and the kit's location, whether indoors or outdoors.

In some embodiments, In some embodiments, the signal from the kit can be received by other devices, such as mobile phones equipped with an application configured to receive signals via Bluetooth or other receivers, such as a network of antennas distributed across locations like a university campus. Upon reception, these devices can trigger the process of notifying emergency services and assist in locating the kit.

Location determination may be achieved by identifying which mobile phone received the signal or by pinpointing the location of the mobile phone or the nearest antenna. Additionally, triangulation can be employed as a method for more accurate location tracking, utilizing multiple devices that receive the signal. The circuit on the case may be configured to send an alert via Bluetooth or another type of signal to initiate the emergency response process.

To enhance its effectiveness, the kit may be configured to send alerts to a predefined contact list, which may include EMS, caregivers, family members, local community responders, or other critical contacts. This contact list may be customized via a companion mobile app or web interface, allowing for easy updates and management of contacts. In addition to notifying EMS, the system may be programmed to alert multiple contacts simultaneously, ensuring rapid and coordinated response efforts. Various alert methods, including, but not limited to, SMS, email, and app-based notifications, may be supported, providing multiple ways to ensure that alerts are promptly received. SMS alerts may offer a direct and immediate way to reach mobile phones, while email notifications may include detailed information such as the kit's GPS coordinates, time of activation, and any other relevant data. App-based notifications may provide real-time tracking, allowing recipients to monitor the kit's location and status continuously. Additionally, the app may provide instructions or guidance on how to assist the individual in distress, bridging the time gap until professional responders arrive.

### Location Tracking and Motion Detection

The kit may integrate advanced location services, such as, but not limited to, GPS, GLONASS, Galileo, or BeiDou, to provide precise, real-time location data, enabling responders to quickly and accurately locate the kit and the individual in need. This approach ensures robust location tracking, reducing signal loss and improving the speed of acquiring a location fix, even in challenging environments like urban canyons or remote areas. Additionally, the system may include accelerometers or other advanced motion sensors, such as gyroscopes, to detect sudden movements, impacts, or orientation changes that may indicate an emergency. These sensors continuously monitor for abrupt changes and can trigger the transmission of an alert signal if specific thresholds are exceeded, such as a fall or a violent shake, providing an additional layer of automatic emergency detection.

### Modular Design and Power Management

In some embodiments, the closed circuit may be housed by a compact board or pad with an adhesive backing (for attachment to the outside of the case), integrated directly into the case to facilitate easy assembly during manufacturing and ensure that the closed circuit remains securely in place throughout transport and use. The board or pad may be designed with a perforated seam that allows it to break into two pieces along the seam, providing a controlled and easy separation. The adhesive board or pad may have a flexible backing such as paperboard and the backing can carry the closed circuit. The case can be adapted to have the opening of the case to cause a mechanical operation on the board or pad (attached to the case) wherein the operation changes the configuration of the circuit to open a portion of the circuit, in response to which the circuit sends a wireless signal. The board or pad may feature dedicated mounting points for each electronic component, including, but not limited to, the microcontroller, communication module, GPS receiver, power management circuit, and motion sensors. This modular design not only simplifies assembly but also enhances durability by protecting components from physical shocks and vibrations.

A reliable power source, such as a long-life lithium-ion or lithium-polymer battery, may be integrated into the kit to operate elements responsible for a signal transmission, e.g., a closed circuit or an additional circuit. These batteries may be selected for their high energy density, durability, and ability to maintain charge over extended periods, ensuring the system remains operational when needed. The power source may have sufficient capacity to support multiple transmissions and continuous monitoring for several months or even years, depending on usage patterns. A power management circuit may be included to monitor battery status, regulate energy consumption, and conserve power when the kit is not in use. This circuit may feature low-power modes, automatic shut-off, and energy-efficient components to extend battery life. In some embodiments, the power source may be rechargeable via an external USB port or other standard charging interfaces, providing flexibility for recharging using commonly available chargers.

### User Guidance and Instructional Support

To support users with minimal training, the kit may include a QR code prominently placed on the exterior or interior surface of the case, linking to comprehensive digital instructions. These instructions may comprise a range of multimedia resources, including visual and written guides, interactive tutorials, and instructional videos that demonstrate proper techniques for recognizing the signs of an emergency and administering the appropriate response. The QR code may be easily scanned using any standard smartphone or tablet, providing immediate access to multilingual content tailored for both laypersons and professionals. This feature ensures that all users, even those without prior experience, can quickly obtain critical, step-by-step information in real time during an emergency, thereby improving the likelihood of a timely and correct response, potentially saving lives.

### Example Embodiments

The following example embodiments of the portable emergency response kit are provided for the purpose of understanding the inventive concepts and should not be construed as limiting the scope of the disclosure. The configurations, components, and features shown in the figures represent only some of the possible implementations of the disclosure and do not encompass all variations or modifications that may fall within the spirit and scope of the disclosed subject matter.

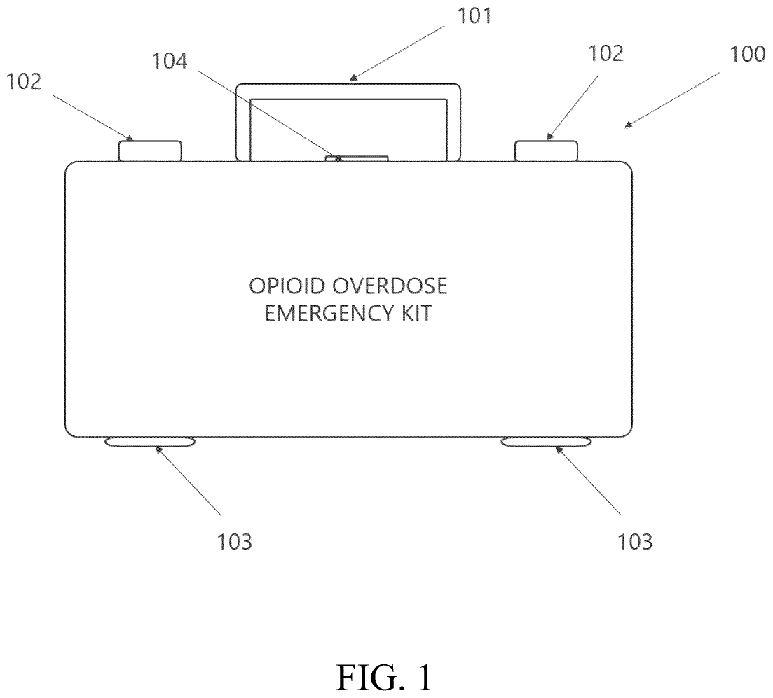

FIG. 1 is a schematic diagram illustrating an exemplary embodiment of the portable opioid overdose emergency response kit 100 in its closed state. The kit is designed to provide a quick response in medical emergencies, such as opioid overdoses, and is housed in a clear plexiglass box with a handle 101 for easy carrying. The clear material allows for visual confirmation of the kit's contents, ensuring that they are visible and ready for use. The case features external clips or locking mechanisms 102. Hinges or similar mechanisms 103 connect the upper compartment 108 and lower compartment 107, allowing the case to open and close smoothly.

The kit includes a closed circuit 104 that is configured to activate a signal transmission when the case is opened. This mechanism, for example, in the form of a safety seal that helps prevent unauthorized access and to keep the contents secure, breaks the seal and an electrical circuit upon opening, automatically notifying emergency medical services (EMS) or predefined contacts of the kit's location. In some embodiments, the closed circuit, upon opening, may send a signal to an additional circuit to notify emergency medical services (EMS) or predefined contacts of the kit's location. The location of the closed circuit 104 can vary within the kit, provided that it is positioned in a way that ensures the opening of the case triggers the disruption or opening of the circuit. The kit may also come with wall mount and mounting hardware, allowing it to be securely installed in accessible locations for quick use during an emergency.

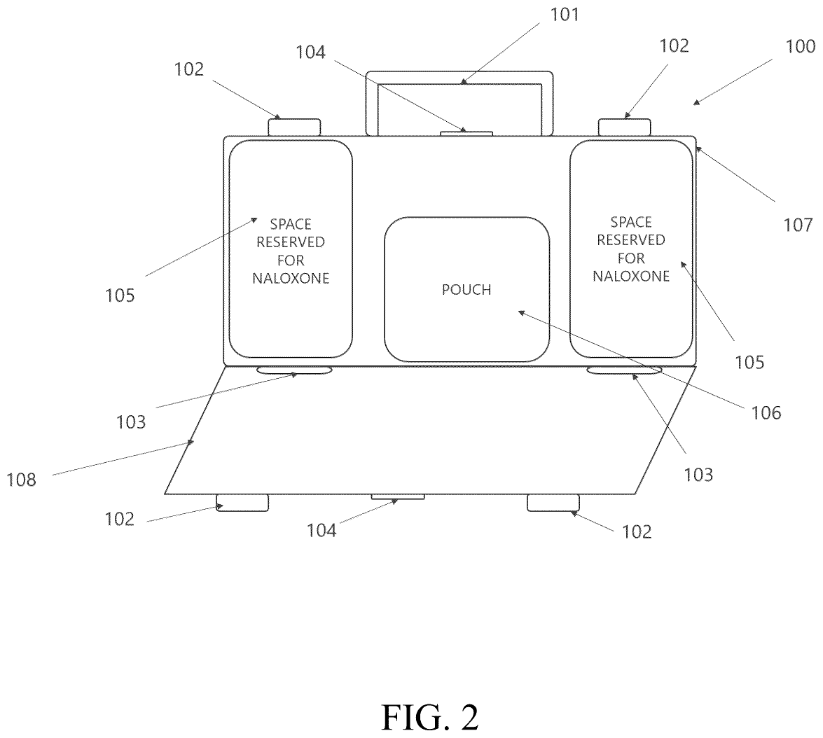

FIG. 2 is a schematic diagram illustrating an exemplary embodiment of the portable opioid overdose emergency response kit 100 in its open state. In this view, the upper compartment 108 and lower compartment 107 are visible, connected by hinges 103 that allow the case to open easily. Inside, designated spaces 105 are reserved for storing opioid overdose treatment medications, such as naloxone, although these medications are not depicted in this drawing. The kit also includes a CPR kit 106, which contains an adult/child CPR mask with a one-way valve and filter, nitrile gloves, and an antiseptic wipe. These items are intended to provide basic support in emergencies, alongside the opioid overdose response tools.

The closed circuit 104 is shown in the open state, illustrating how it is triggered to send a signal to notify emergency medical services (EMS) or predefined contacts of the kit's location when the case is opened. The closed circuit may be in the form of a tamper-evident seal to help maintain the security of the case and its contents. The external clips or locking mechanisms 102 also play a role in securing the case, which may include safety seals. The handle 101 provides easy portability, allowing the kit to be transported quickly to the location where it is needed.

These figures provide an example of how the portable emergency response kit can be configured to offer immediate medical assistance and automated emergency alerts. The inclusion of both opioid overdose treatment supplies and a CPR kit provides a range of response options for different types of medical emergencies. The tamper-evident features, clear material, and mounting options help ensure that the kit is secure, visible, and accessible in various settings.

### Additional Embodiments and Features

In some embodiments, the portable emergency response kits may incorporate a range of additional features designed to enhance their module, usability, and reliability in critical situations. To ensure the integrity and readiness of the kit's contents, the kits may be equipped with tamper-evident seals. These seals may be crafted from specialized materials that visibly deform or display an unmistakable indication—such as color changes, tearing, or revealing hidden text—if the case has been opened or tampered with. This feature provides an added layer of security and assurance, giving users confidence that the kit remains fully stocked, secure, and ready for immediate use in emergencies. The tamper-evident seals may be further customized with unique serial numbers or barcodes, enabling organizations or responders to track and authenticate the kits as part of broader inventory management and quality control processes.

The kits may also be designed with ergonomic features that enhance ease of use and handling during emergency situations. For example, they may be equipped with easy-grip handles contoured to fit comfortably in the hand, even when wearing gloves or in wet conditions. These handles may be complemented by quick-release latches that allow the case to be opened swiftly and securely with minimal effort, facilitating rapid access to the life-saving equipment inside. The overall form factor of the case may be compact and lightweight, making it easy to carry, store, or transport. Additionally, the cases may be constructed with waterproof or water-resistant materials and seals to protect their contents from exposure to moisture, rain, or other environmental hazards. This waterproofing feature ensures that the medication or devices inside remain functional and safe, even in adverse conditions, such as heavy rain, flooding, or exposure to water during rescue operations.

In some embodiments, the kits may include an audible alarm or LED indicator that activates when the case is opened or when the signal transmission is triggered. The audible alarm may produce a loud, distinctive sound, while the LED indicator may emit a bright, flashing light, both of which serve to provide a local alert to nearby responders or individuals. These alerts can draw immediate attention to the emergency situation, prompting swift action from those in the vicinity. The alarm and indicator may be especially useful in crowded or noisy environments, where visual or auditory cues can quickly notify bystanders, security personnel, or trained responders that immediate assistance is required. Additionally, when the case is opened or signal transmission is triggered, the kits may provide verbal instructions to guide the user in using the kit without prior training or knowledge.

To further enhance usability and convenience, the kits may come with a range of optional accessories designed to facilitate transport, storage, and deployment. For instance, they may include adjustable carrying straps that allow the kit to be worn over the shoulder or across the body, freeing up the hands for other tasks. Mounting brackets may also be provided, allowing the kits to be securely affixed to walls, vehicles, or other fixed structures, ensuring they are easily accessible in a variety of settings, such as public spaces, workplaces, or homes. Additionally, protective pouches or cases may be offered to safeguard the kit during transport or storage, protecting it from dust, debris, and physical damage.

These features collectively enhance the portability, durability, and versatility of the kits, making them adaptable to various emergency scenarios and environments. The combination of tamper-evident security measures, ergonomic design elements, robust communication capabilities, local alert mechanisms, and practical accessories ensures that the portable emergency response kits are not only effective in delivering timely assistance but also user-friendly and adaptable to the needs of different users and situations. This comprehensive approach maximizes the kits' readiness and accessibility, ultimately improving the chances of successful interventions and saving lives in critical situations.

Overall, the portable emergency response kit combines precise location tracking, advanced motion detection, robust power management, comprehensive user guidance, and automatic alert mechanisms, ensuring that it is both user-friendly and highly effective in delivering timely assistance during emergencies. Whether used for opioid overdose treatment, as an AED kit, or for other conditions, this versatile solution provides a critical link between the kit's deployment and the arrival of emergency assistance, enhancing its reliability and accessibility in life-saving interventions.

The scope of the claims should not be limited by the preferred embodiments set forth in the examples but should be given the broadest interpretation consistent with the description as a whole. Various modifications, adaptations, and alternative implementations may be made without departing from the scope and spirit of the disclosure.

It should be understood that the method and apparatus described herein are not limited to the specific forms or arrangements of parts described and illustrated. Various changes and modifications may be made within the scope of the disclosure. For example, the cleaning and/or treatment solution compositions, the materials used for the substrate, and the specific configurations of the cutting mechanisms can all be varied to suit particular requirements and environments. The disclosure encompasses all alternative, modified, and equivalent methods and systems as they fall within the spirit and scope of the following claims.

What is claimed is:

1. A portable opioid overdose response kit comprising:
a case having a compartment configured to contain an opioid overdose treatment medication;
a breakable closed circuit configured to be triggered upon opening the case, wherein opening the case breaks the circuit, which triggers the kit to send a signal to a communication device external to the kit.

2. The kit of claim 1, wherein the compartment contains the opioid overdose treatment medication.

3. The kit of claim 1, wherein opening the case breaks the circuit, which triggers the kit to send a signal to notify emergency medical services (EMS) or predefined contact list.

4. The kit of claim 1, wherein the case is made of transparent material that allows visual confirmation of contents of the case.

5. The kit of claim 1, further comprising a QR code linked to instructions for administering the opioid overdose treatment medication.

6. The kit of claim 1, further comprising a GPS module to transmit the location of the kit.

7. The kit of claim 1, further comprising a battery power source.

8. The kit of claim 1, wherein the case comprises an upper compartment and a lower compartment, and the breakable closed circuit is configured to be triggered upon separating the upper compartment from the lower compartment, wherein separating the compartments breaks the circuit, and the broken circuit is triggered to send the signal to the communication device external to the kit.

9. The portable opioid overdose response kit of claim 1, wherein the circuit is housed by a board or pad with an adhesive backing, which is integrated into the case.

10. A portable Automated External Defibrillator (AED) kit comprising:
a case having a compartment configured to contain AED;
a breakable closed circuit configured to be triggered upon opening the case, wherein opening the case breaks the circuit, which triggers the kit to send a signal to a communication device external to the kit.

11. The kit of claim 10, wherein the compartment contains an Automated External Defibrillator (AED).

12. The kit of claim 10, wherein opening the case breaks the circuit, which triggers the kit to send a signal to notify emergency medical services (EMS) or predefined contact list.

13. The kit of claim 10, wherein the case is made of transparent material that allows visual confirmation of the contents of the case.

14. The kit of claim 10, further comprising a QR code linked to instructions for operating the AED.

15. The kit of claim 10, further comprising a GPS module to transmit the location of the kit.

16. The kit of claim 10, further comprising a battery power source.

17. The kit of claim 10, wherein the case comprises an upper compartment and a lower compartment, and the breakable closed circuit is configured to be triggered upon separating the upper compartment from the lower compartment, wherein separating the compartments breaks the circuit, and the broken circuit is triggered to send the signal to the communication device external to the kit.

18. The kit of claim 10, wherein the circuit is housed by a board or pad with an adhesive backing, which is integrated into the case.

19. A portable emergency response kit comprising:
a case having an upper compartment and a lower compartment,
a breakable closed circuit configured to be triggered upon separating the upper compartment from the lower compartment, wherein separating the compartments breaks the circuit, which triggers the kit to send a signal to a communication device external to the kit.

20. The kit of claim 19, wherein the circuit is housed by a board or pad with an adhesive backing, which is integrated into the case.
